(19) United States
(12) Patent Application Publication
(10) Pub. No.: US 2022/0125425 A1
(43) Pub. Date: Apr. 28, 2022

(54) PRE-FORMED SURGICAL KNOT LOADING SYSTEMS AND METHODS OF USE

(71) Applicants: Smith & Nephew, Inc., Memphis, TN (US); Smith & Nephew Orthopaedics AG, Zug (CH); Smith & Nephew Asia Pacific Pte Limited, Singapore (SG)

(72) Inventor: Benjamin Michael Hall, Roslindale, MA (US)

(21) Appl. No.: 17/509,480

(22) Filed: Oct. 25, 2021

Related U.S. Application Data
(60) Provisional application No. 63/105,519, filed on Oct. 26, 2020.

(51) Int. Cl. A61B 17/04

(52) U.S. Cl. CPC A61B 17/0482; A61B 2017/0488; A61B 17/0485; A61B 17/0401

(57) ABSTRACT

Disclosed herein is a loading tray for managing a flexible member construct. The flexible member construct includes a pre-formed knot with a first and second limb extending therefrom. The loading tray includes a base and a knot loop housing flexibly coupled to the base. The knot loop housing has an outer peripheral surface for holding at least one loop of the pre-formed knot in a dilated configuration. The knot loop housing includes a channel to receive an instrument shaft therethrough and thereby place an instrument shaft through the at least one loop of the pre-formed knot. The knot loop housing includes a free end, such that tension from either a limb of the flexible member construct and/or instrument shaft draws the at least one loop onto the instrument shaft.

### CROSS-REFERENCE TO RELATED APPLICATIONS

This application claims benefit to and incorporates by reference in its entirety, U.S. Provisional Patent No. 63/105,519, filed Oct. 26, 2020; titled “PRE-FORMED SURGICAL KNOT SYSTEMS AND METHODS FOR USE WITH SUTURE PASSERS”.

### FIELD

The present disclosure relates to pre-formed knots in flexible members for surgical use and methods of managing these pre-formed knots. These pre-formed knots may cooperate with suture passers to repair soft tissue.

### BACKGROUND

Arthroscopic soft tissue repair may involve passing a flexible member through the soft tissue and then securing this flexible member. The flexible member may be a suture or suture tape for example. A suture passer may pass the flexible member through the soft tissue. The flexible member may then be secured to the soft tissue with a knot. In arthroscopic hip capsule closure, a plurality of sutures may be required, with a plurality of knots.

Knots may be formed by the surgeon, which may be a sliding knot, and may be formed external to the patient body. Forming and managing the knot can be a time-consuming process and add complexity to the surgery. Therefore, there is a need for an improved system that addresses the issues listed.

### SUMMARY

Described herein are various improvements in methods and devices associated with repairing soft tissue with a flexible member construct. A flexible member may include a suture, braided material, cable, wire, ribbon or suture tape. Improved methods may include a means of managing assembly of a flexible member construct including a pre-formed knot to a surgical instrument for use during arthroscopic tissue repair. The surgical instrument may be a suture passer. The pre-formed knot may be provided in a partially constructed configuration, that is incomplete as a sliding/locking knot. In its complete configuration, the pre-formed knot may form a sliding locking knot. The method disclosed herein may include a means of changing the configuration of the preformed knot to the sliding locking knot, during the tissue repair.

Disclosed herein is a loading tray for managing a flexible member construct that includes a pre-formed knot. Managing may include managing the steps of assembly to a surgical instrument. The pre-formed knot includes at least one loop, a first limb and a second limb. The loading tray includes a base and a knot loader. The knot loader is hingedly coupled to the base and is generally a 3-dimensional construct that stands proud of the base. The knot loader has an outer peripheral surface that may include a plurality of angularly offset surfaces, configured to hold the at least one loop of the pre-formed knot in a dilated configuration. The pre-formed knot is provided in an unlocked configuration, such that tension on the limbs may alter a loop size. The pre-formed knot may be provided in a partially pre-formed configuration. Stated another way, the pre-formed knot defines a complete configuration for tissue repair that is preferably a sliding locking knot, and the pre-formed knot may be provided incomplete as a sliding locking knot, but partially pre-formed such that additional threading steps of the flexible member through the pre-formed knot as provided completes the pre-formed knot and thereby changes it to the complete configuration. The knot loader also includes a channel extending therethrough, sized to receive an instrument shaft therein. Inserting the instrument shaft through the channel places the instrument shaft concomitantly through the at least one loop of the pre-formed knot.

In some particular embodiments, the knot loader is hingedly coupled at a first end of the knot loader and external forces from the instrument shaft may pivot the knot loader around the first end and draw the at least one loop over a free end of the knot loader and onto the instrument shaft. This removes the at least one loop and pre-formed knot (incomplete or complete) from the knot loader. In some particular embodiments, the base defines a thin planar element. In some particular embodiments, the loading tray is entirely formed from a single unibody. This single unibody may be formed of cardboard, paper, cork or a polymer. In some particular embodiments, the loading tray is formed of a single card sheet and the knot loader includes angularly offset portions, formed by bending the single card sheet. The bends may orient side surfaces of the knot loader perpendicularly to the loading tray base. The knot loader may be hingedly coupled via a living hinge. The loading tray may include a first and a second slot for holding a first and a second limb of the flexible member construct respectively.

An example method of arthroscopically repairing a tissue is also disclosed. The method may use a suture passer and a flexible member construct assembled to a loading tray. The flexible member construct includes at least one flexible member provided with a pre-formed knot, the preformed knot being in an unlocked, slideable configuration with at least one dilated loop. The pre-formed knot may be provided as an incomplete knot. The flexible member construct includes a first and second limb extending from the pre-formed knot. The at least one dilated loop is assembled around a housing of the loading tray. The method may include passing a distal end of the suture passer through a channel of the housing, and thereby threading the suture passer through the at least one dilated loop. A first limb may then be operatively coupled with a working end of the suture passer. The at least one dilated loop may be removed from the housing and onto a shaft of the suture passer. The first limb may then be passed through the tissue in need of repair, with the suture passer, while the at least one dilated loop remains on the shaft of the suture passer. The at least one dilated loop may be removed from the suture passer to thread the first limb through the dilated loop. Threading the first limb through the dilated loop may complete the knot and form a sliding locking knot. The at least one dilated loop may be reduced to repair the tissue.

Some particular methods may include removing the first limb from a first slot of the loading tray to couple the first limb with the working end of the suture passer. The second limb may be removed from a second slot of the loading tray, the second slot being different from the first slot. The second limb may be removed after passing a distal end of the suture passer through the channel. Tension may be applied on the second limb to reduce the lumen size of the at least one dilated loop and engage the at least one dilated loop around the suture passer. This may secure the at least one dilated loop around the suture passer shaft. In some particular methods, the first limb may be coupled to the working end after reducing the at least one dilated loop to a reduced lumen configuration around the suture passer. Once coupled to the working end, the first limb may be passed through a first portion of tissue and a second portion of tissue with the suture passer to form a repair loop through the tissue. The at least one dilated loop may then be removed from the passer shaft. With the first limb coupled to the working end, the at least one dilated loop may be slid distally, to thread the first limb through the at least one dilated loop. This step may complete the repair loop and change the pre-formed knot to a complete knot that is a locking sliding knot. The at least one dilated loop may be in the reduced lumen configuration, during this step. Removing the at least one dilated loop may reduce a loop size of the repair loop. The at least one dilated loop may be further reduced to a cinching loop configuration to fix the repair loop and repair the tissue.

An example method of assembling a flexible member construct with an instrument shaft is also disclosed herein. The flexible member construct may be provided with a pre-formed knot with at least one dilated loop and a first and second limb. The pre-formed knot may be incomplete as a sliding and locking knot as provided. The method may include inserting an instrument shaft through the dilated loop, the dilated loop wrapped around a knot loader of a loading tray. The dilated loop may then be slid over a free end of the knot holder and onto the instrument shaft.

In some particular methods, at least one of the first and second limbs may be tensioned to draw the at least one dilated loop over the free end of the knot holder. In some particular methods, the suture passer may be lifted away from the knot loader to pivot a hinge of the knot loader and draw the dilated loop over the free end of the knot holder. Tensioning one of the first or second limb may tighten the at least one dilated loop around the instrument shaft. The method may include operatively coupling the first limb to a working end of the suture passer. The first and second limb may both be operatively coupled to the loading tray and therefore the method may include removing the first limb from the loading tray and operatively coupling the first limb to a working end of the suture passer, and also removing the second limb from the loading tray and pulling on the second limb to reduce the at least one dilated loop. The instrument shaft may be inserted through the at least one dilated loop by inserting the instrument shaft through a channel of the knot loader. The at least one dilated loop of the pre-formed knot may be drawn over the first limb, to change the preformed knot to a complete configuration and form a locking knot. This may form a Weston knot.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The disclosure will be more fully understood by reference to the detailed description, in conjunction with the following figures, wherein:

FIG. 1A illustrates an example locking and sliding knot in a complete configuration;

FIG. 1B illustrates the example locking and sliding knot in an incomplete or partially formed configuration;

FIG. 2 illustrates an example embodiment of a loading tray, assembled with a flexible member construct, in accordance with this disclosure;

FIG. 3 illustrates an example method of forming a loading tray, in accordance with this disclosure;

FIG. 4 illustrates another example embodiment of a loading tray, in accordance with this disclosure;

FIGS. 5A-5D illustrate a method of loading a flexible member construct onto a surgical device with a loading tray, in accordance with this disclosure;

FIGS. 6A-6C illustrate a method of repairing tissue with a flexible member construct, in accordance with this disclosure; and

FIG. 7 illustrate another example embodiment of a loading tray, in accordance with this disclosure.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open ended and include the listed parts and can include additional parts that are not listed. “And/or” is open-ended and includes one or more of the listed parts and combinations of the listed parts. Use of the terms “upper,” “lower,” “upwards,” and the like is intended only to help in the clear description of the present disclosure and are not intended to limit the structure, positioning and/or operation of the disclosure in any manner.

Generally disclosed herein is a means of providing and managing a flexible member construct for repairing tissue, the flexible member construct including a pre-formed knot. The pre-formed knot may be provided partially deconstructed or incomplete, and when completed is configured to be a sliding and locking knot. The preformed knot, whether provided incomplete or complete, may be provided with at least one dilated loop. Managing the pre-formed knot may include a means of maintaining at least one dilated loop of the knot in the dilated configuration for receiving an instrument shaft and/or flexible member limb therethrough. The flexible member construct may be used to repair tissue. For example, the flexible member construct may repair an incision or tear in soft tissue such as hip capsule tissue towards the end of a hip arthroscopy.

The means of providing and managing the flexible member construct may include a “loading tray”. Loading tray is used to define a board, card or panel, cassette, or a molded component for managing a flexible construct assembled thereto and its staged release. Loading tray includes a base with a plurality of protrusions or slots for managing various portions of the flexible member construct. Loading tray is preferably configured to load a pre-formed knot (in a complete or incomplete configuration) onto a shaft of a surgical instrument, for example a suture passing instrument. Some suture passing instruments may include an axially sliding hook, such as the suture passer disclosed in U.S. Pat. No. 10,265,062, commonly owned and herein incorporated by reference in its entirety. Other suture passing instruments may include pivoting jaws, such as the suture passer disclosed in U.S. Pat. No. 9,211,118, commonly owned and herein incorporated by reference in its entirety. Passers with smooth tapered shafts without pivoting jaws may be preferable, such as the axial sliding hook embodiments. This style is absent a plurality of surface topologies inherent with components included with moving jaw suture passers that may catch on the knot loop.

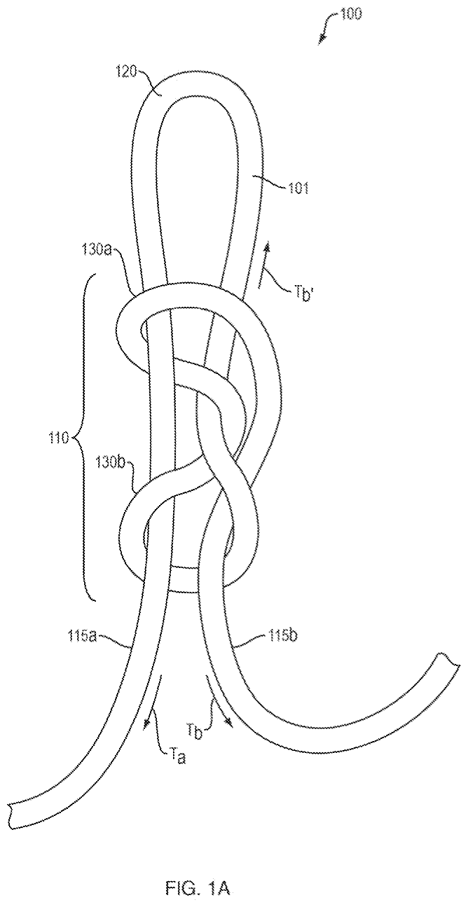

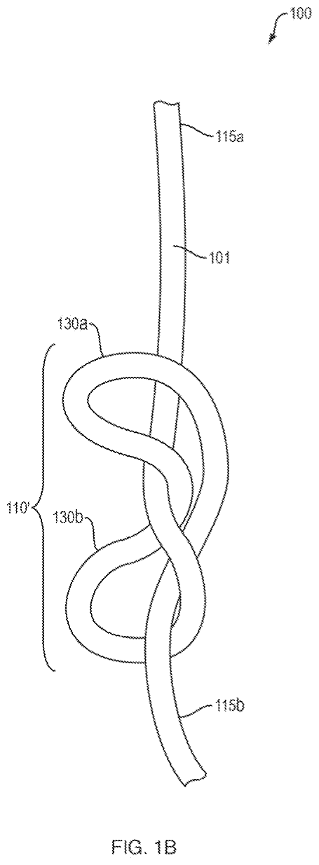

An example flexible member construct 100 is shown in FIG. 1A within a complete sliding locking knot 110. Flexible member construct 100 may include at least one flexible member 101, formed in a pre-formed knot 110 that preferably slides to reduce with a first tension and locks the knot 110 with a second tension. Complete knot 110 may define a first loop 120, two locking loops 130a, 130b, a first limb 115a and a second limb 115b. The example knot shown is a Weston sliding knot. Tension (Tb) on the second limb 115b may reduce the two locking loops 130a, 130b to cinch around limb 115a, and therefore place the knot 110 in a locked configuration, and therefore lock the first loop 120. Of note, counter tension Tb′ may also lock the knot 110. Tension (Ta) on the first limb 115a may reduce the first loop 120 if the knot 110 is not locked.

Flexible member construct 100 may be provided with an incomplete pre-formed knot 110′, illustrated in FIG. 18. With reference to the particular example knot 110, to form a complete pre-formed knot 110, that is a sliding and locking knot, useful for arthroscopic surgery, limb 115a may be looped to form loop 120 and then threaded through the two loops 130a, 130b, to form the knot 110, shown in FIG. 1A. During the tissue repair, the loading tray may cooperate with the surgical instrument to repair the tissue and concomitantly change an incomplete knot 110′ to a completed locking knot configuration (110).

A flexible member construct, such as flexible member construct 100 may be provided assembled to a loading tray. As provided a partial or incomplete Weston knot is shown, although other knot embodiments may include other forms of sliding and locking knot (complete or incomplete) with a reduceable loop. For example, flexible member construct could include a Weise Knot. Loading tray is configured to manage and stage the assembly or release of portions of the flexible member construct 100. For example, the loading tray may provide a means of assembling the loops 130a, 130b around a suture passer shaft. Loading tray may provide a means of holding and selectively releasing limbs 115a, 115b. For example, at least one of the limbs 115a or 115b may be removed and tension applied thereto, to selectively reduce the loops 130a, 130b and engage loops 130a, 130b with the instrument shaft. Limb 115a may be removed and coupled to a suture passer's working end.

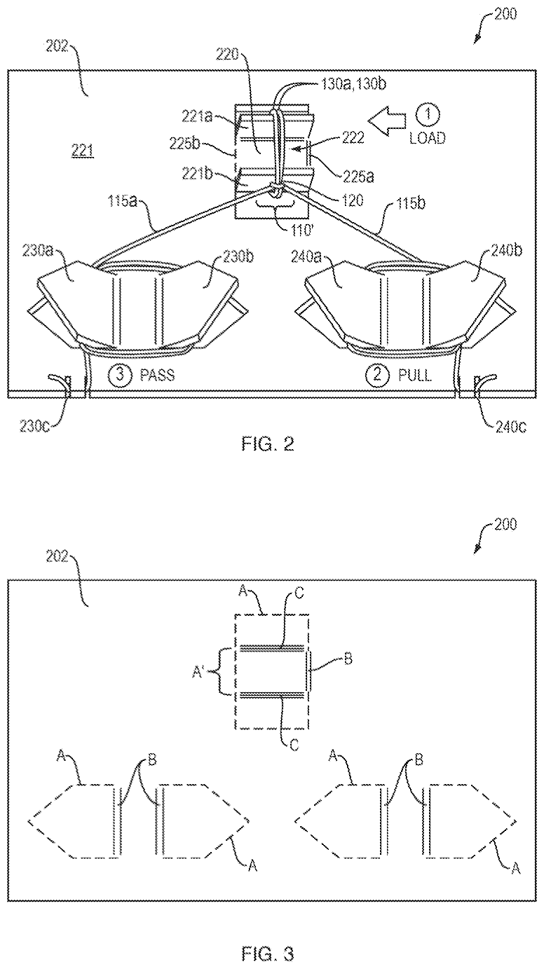

A first example loading tray in the form of a card 200 is shown in FIG. 2. Card 200 may be formed from a single sheet of foldable cardstock. Card 200 may be a thin planar element with a plurality of living hinges or tabs formed therein to manage portions of flexible member construct 100. Card 200 is sufficiently bendable to form functional living hinges and folds, and yet is sufficiently rigid to be self-supportive. Card 200 is sufficiently rigid to hold knot loops 130a,130b in a dilated configuration and form a channel via folds in the card that remain open to receive an instrument shaft therethrough. Card 200 includes a knot loader 220 configured to hold a loop or loops of a knot, such as dilated loops 130a, 130b of incomplete knot 110′. Card 200 may also include a first pair of flexible tabs 230a, 230b for managing the first limb 115a and a second pair of flexible tabs 240a, 240b for managing the second limb 115b. Card 200 may also include indicia, words or markings thereon to aid in staging the release and assembly of the construct 100 onto an instrument shaft.

Knot loader 220 may define a 3-dimensional construct include multiple folded portions of the card 200. Knot loader 220 may stand proud of the planar surface 221 of the card base 202. Loader 220 may define two parallel sides 221a, 221b extending perpendicular to planar surface of card base 202 and substantially parallel to each other. Loops 130a, 130b may encircle and engage outer peripheral surfaces of loader 220. Loops 130a, 130b may encircle the outer peripheral surface and extend under the card base 202. Loops 130a, 130b may be in a dilated configuration, but sized to frictionally engage or cinch around the outer peripheral surfaces of loader 220 and therefore loops 130a, 130b are held in place around the loader 220 in a dilated configuration. Loops 130a, 130b may form a complete 360-degree loop around loader 220. Knot loader 220 may define a channel 222 with three sides. Channel 22 is configured to receiving an instrument shaft therethrough, such that inserting the instrument shaft through the channel 222 concomitantly threads the instrument shaft through the loops 130a, 130b. Inserting the instrument shaft through the channel 222 concomitantly threads the instrument shaft through the loops 130a, 130b mimicking the path limb 15a takes through loops 130a, 130b as illustrates in FIG. 1A. Loader 220 may define three sides of channel 222. Channel 222 may be formed by bends oriented to form three internal surfaces of a square or rectangular shaped channel in cross section. Cross section may be uniformly shaped along its length, as shown. In other embodiments, cross section may be tapered (not shown). Channel 222 is sized to receive instrument shafts between 2-10 mm in diameter. Channel 222 may define a central axis that is parallel to and offset from planar surface of base 202. Loader 220 may orient the loops 130a, 130b to lie substantially on a plane that extends perpendicularly from the planar surface 221.

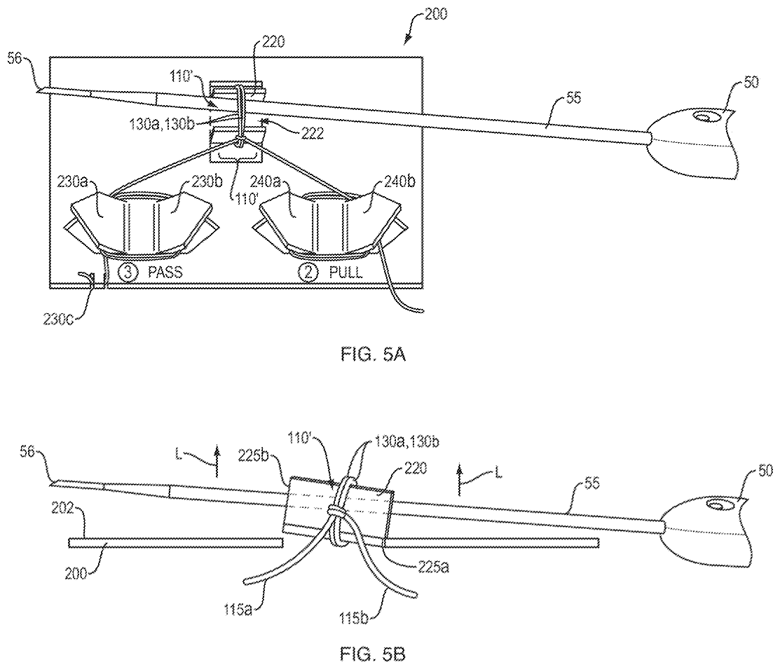

Loader 220 may define a coupled end 225a that may be a living hinge such that the loader 220 may pivot about coupled end 225a. Pivoting may facilitate easier loading of the loops 130a, 130b from the loader 220 and onto the instrument shaft. Loader opposite end 225b may be a free end permitting removal of the loops 130a, 130b from loader 220. In some embodiments, end 225b may be provided loosely coupled to card 200. For example, end 225b may include perforations that provides the loader 220 in an aligned and temporarily fixed position, during initial manipulation. End 225b may then be readily released (perforations may be torn) to be detached upon external forces thereon. For example, an instrument shaft threaded through the channel 222 may be slightly lifted or angled to tear the perforations. Loader 220 may include a saddle (shown in FIG. 4) along top edges of sides 221a, 221b to inhibit loops 130a, 130b from inadvertently slipping off the loader 220.

First limb 115a may extend from incomplete knot 110′ and wrap around tabs 230a and 230b to manage the length of limb 115a. The end of limb 115a may be inserted through a slot 230c at edge of card 200 for selective removal. In a similar fashion, second limb may extend from incomplete knot 110′ and wrap between tabs 240a and 240b to manage the length of limb 115b. The end of limb 115b may be inserted through a slot 240c at edge of card 200 for selective removal.

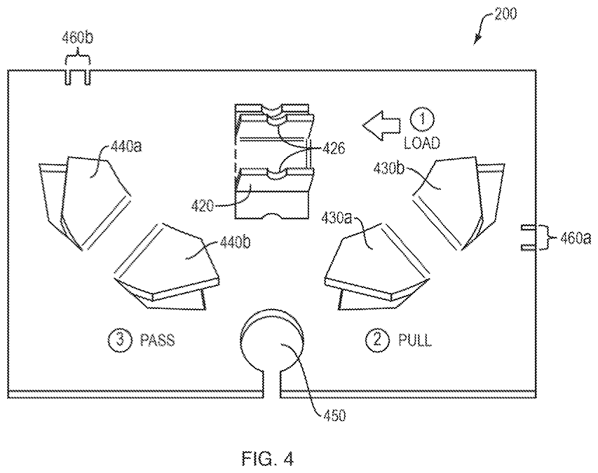

FIG. 3 illustrates a method of forming loading card 200. Lines A indicate cut lines through thickness of card 200. Lines A′ may be partially cut to form perforations. Lines B indicate coupled and folded portions of the card 200 that may form living hinges in that they flex during assembly and disassembly of flexible member construct 100. Lines C indicate fold lines that are folded during formation of the loader 220 to define channel 222.

FIG. 4 illustrates another example loading card 400, similar to card 200, with the flexible member construct removed. Card 400 may be formed from a single sheet of foldable card or cork. Cork may require scoring to define fold or living hinge lines. Card 400 defines a thin planar element with a plurality of living hinges or tabs formed therein to manage portions of flexible member construct 100. Card 400 is sufficiently bendable to form functional living hinges and folds, and yet is sufficiently rigid to be self-supportive, hold a knot loops 130a, 130b in a dilated configuration and form a channel by folds in the card that remain open to receive an instrument shaft therethrough. Card 400 includes a knot loader 420, similar to loader 220 and includes a saddle 426 formed in at least one top edge of loader (both sides shown). Saddle 426 defined a dip or groove configured to inhibit loops 130a, 130b from inadvertently slipping off the loader 420.

Card 400 may also include a plurality of flexible tabs 430a, 430b, 440a and 440b that operative in a similar fashion to tabs 230a, 230b, 240a and 240b for managing the limbs of the flexible member construct. Tabs may be angled at a non-orthogonal angle relative the loader 420 to save space and allow for a smaller profile card 400 relative to card 200. Card 400 may include pairs of slots 460a and 460b extending from an outer peripheral edge of card 400. A terminal end of first limb 115a may engage a first slot pair 460a and form a loose loop between the pair 460a, the loop providing a means to grasp and release the limb terminal end from the card 400. The terminal end of second limb 115b may engage a second slot pair 460b in a similar manner.

Card 400 may include an opening 450 configured to engage a shaft of a surgical tool. During arthroscopic procedures, several tools are used that remain stationary or inert during a substantial portion of the procedure. Non-limiting example tools include a surgical positioning arm, a cannula or a retractor. Opening 450 may frictionally engage a shaft of such a surgical tool, such that hold the card 400 may be released, freeing up a surgeon or surgical assistant hands for other portions of the procedure.

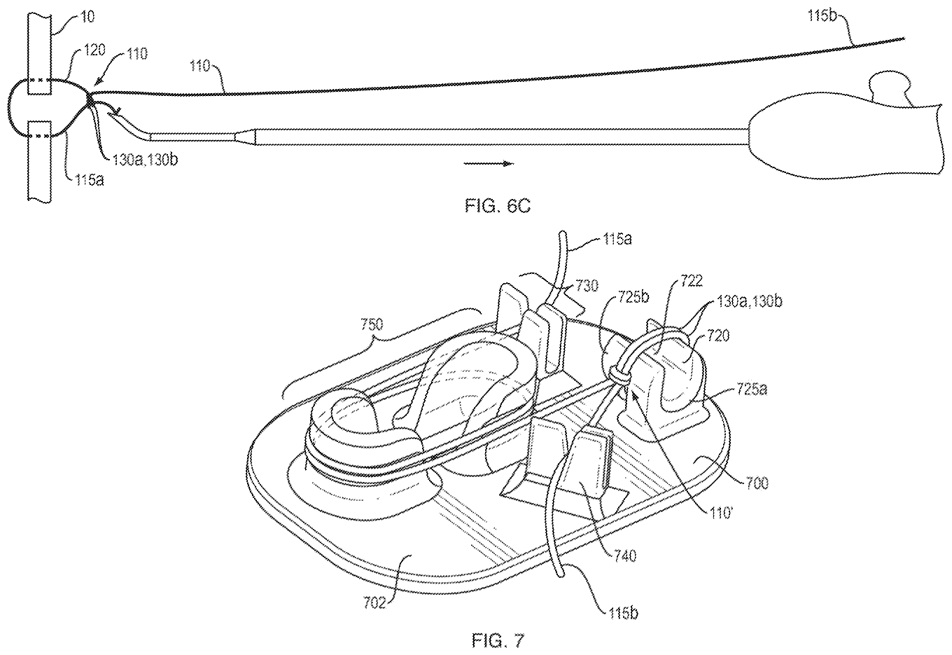

FIG. 7 illustrates another example loading tray 700 that may be molded and therefore may be formed from a polymer. Tray 700 may be a unibody, forming a single shot molded polymeric based component. Tray 700 is formed from a material that may be flexible in some locations. Tray 700 may include a planar base with a plurality of living hinges or flexible slots to manage portions of flexible member construct 100. In this embodiment, some elements such as the knot loader 720 may be formed during molding and therefore is not folded or bent during manufacture. Dilated loops 130a, 130b may wrap around external surfaces of loader 720, loader 720 having a molded channel 722 to receive an instrument shaft therethrough. Tray 700 may also include a plurality of flexible projections 730 that may extend perpendicularly from base 702 for managing the first limb 115a and a second plurality of flexible projections 740 for managing the second limb 115b.

Loader 720 may define a 3-dimensional construct extending from base 702. Loader 720 may include a convex curved outer surface to engage loops 130a, 130b. loops 130a, 130b may be in a dilated configuration, but sized to frictionally engage or cinch around the outer convex curved surface of loader 720. Outer surface may include a protrusion or circumferential channel (not shown) to inhibit inadvertent release of the loops 130a, 130b from loader 720, similar to saddle 426. Loader channel 722 may receive an instrument shaft 55 therethrough, such that inserting the shaft 55 through the channel 722 concomitantly threads the instrument shaft 55 through the loops 130a, 130b. Loader 720 may define three sides of a channel 722. Channel 722 is sized to receive instrument shafts between 2-10 mm in diameter. Channel 722 may define an axis that is parallel to and offset from planar surface of base 702, such that instrument shaft 55 may lie parallel to base 702 while inserted through channel 722. Loops 130a, 130b may each form complete (360 degrees) loops around the loader 720.

Loader 720 may define a coupled end 725a that may be flexible and thereby define a living hinge. Similar to previous embodiments, loader 720 may pivot about coupled end 725a. Pivoting may facilitate easier removal of the loops 130a, 130b from the loader 720 and onto the instrument shaft 55. Loader opposite end 725b may be a free end permitting removal of loops 130a, 130b from loader 720.

First limb 115a may extend from knot 110′ and extend through tabs 730 to manage the length of limb 115a. First limb 115a may also wrap around spool 750. Spool 705 may include a pair of U-shaped projections extending from surface 702, that face each other. Limb 115a may wrap around an external surface of both projections. Limb 115a may be longer than limb 115b to feed arthroscopically through the tissues without undue tension on the construct 100. In a similar fashion, second limb may extend from knot 110′ and extend through tabs 740 to manage the length of limb 115b.

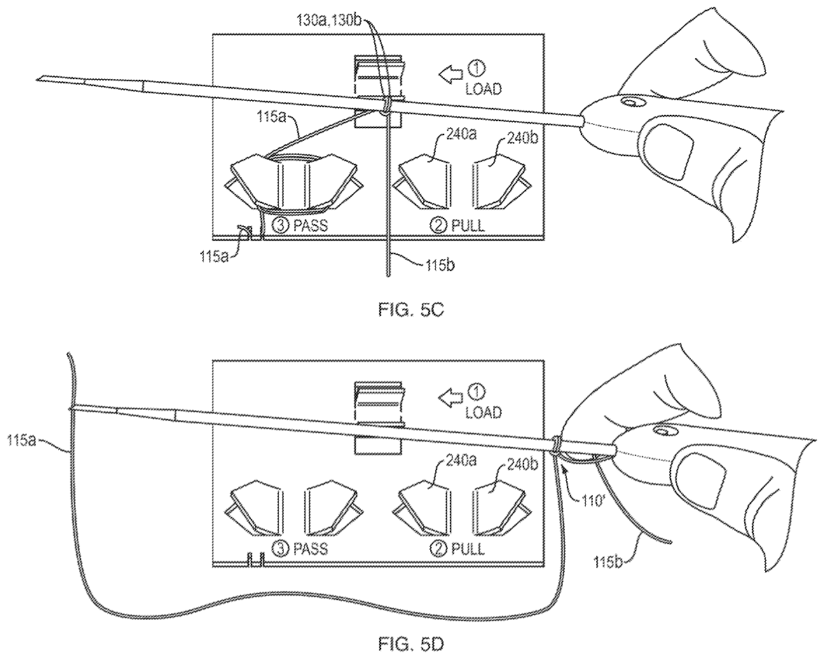

FIGS. 5A-5D illustrate a method of assembling flexible member construct 100 onto an example instrument shaft 55. Instrument 50 may be a suture passer with a shaft 55 and working distal end 56. Example loading tray 200 is illustrated in this example method, although the method may be similar for other tray embodiments 400 or 700. Working end 56 of instrument 50 may be inserted through channel 222 of card's knot loader 220 and thereby threaded through dilated loops 130a, 130b of pre-formed knot 110′. Instrument 50 may then be lifted in direction of arrows (L) to slide loops 130a, 130b over loader free end 225b. Alternatively, or in addition to lifting the instrument 50, card 200 may be pulled away from instrument 50 to slide loops 130a, 130b over loader free end 225b. Loader 220 may rotate about a living hinged end 225a while removing loop 130a, 130b (FIG. 5B). Loops 130a, 130b may be removed with the two limbs 115a, 115b remaining engaged around tabs 230a, 230b, 240a, 240b respectively. In some example methods, second limb 115b may be uncoiled and thereby removed from tabs 240a, 240b and tension applied thereto, to help draw the loops 130a, 130b off loader 220. Once unloaded, tension on the second limb 115b reduce loops 130a, 130b and tighten them around shaft 55 (FIG. 5C). Reducing the loops 130a, 130b helps retain them on shaft 55. Once unloaded onto the shaft 55, a working end 56 of suture passer may engage first limb 115a (FIG. 5D). Working end 56 may capture and remove a looped end of first limb 115a formed at slots 230c. In some particular methods, lifting the instrument may first tear a perforated knot loader end 225b before the loader 220 rotates to unload the loops 130a, 130b. The method may include first engaging the tray 200, 400, 700 to a shaft of a stationary surgical tool and assembling the flexible member construct 100 to the instrument 50 while the card 200, 400, 700 is frictionally held by stationary surgical tool.

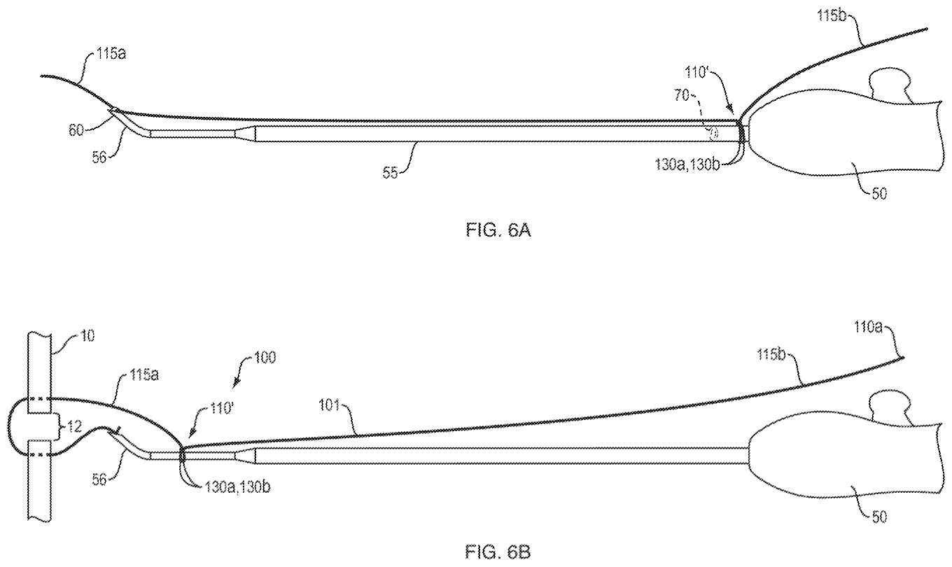

The method may continue to repair a tear or incision in a tissue using the suture passer 50 with the flexible member construct 100 assembled thereto. An example method of repair is illustrated in FIGS. 6A-6C. FIG. 6A illustrates suture passer instrument 50 with the loops 130a, 130b disposed around shaft 55. First limb 115a is operatively coupled to working end 56. Suture passer may define a needle 60 with a tissue piercing end and an axially moving hook that selectively grabs and releases limb 115a to place limb 115a through the tissue. Suture passers with axially moving hooks may include the ACCUPASS Direct, offered by Smith and Nephew. Suture passer 50 may be similar to the instruments disclosed in U.S. Pat. No. 10,265,062, commonly owned and herein incorporate by reference in its entirety. FIG. 6B illustrates the limb 115a having been serially passed through tissue 10 twice, one each side of a tissue tear or incision 12. An example tissue includes leaflets of a hip capsule for example. With sliding hook recessed within the shaft working end 56, knot 110′ may be slid distally along shaft 55 and working end 56 and then over limb 115a. This forms a complete knot 110, by threading limb 115a through loops 130a, 130b. This forms repair loop 120. Having the hook recessed, reduces any snagging of the knot 110′ as it slides distally over the working end 56. Needle 60 therefore provides a smooth, continuous outer surface for sliding therealong. Knot 110′ and loops 130a, 130b may slide distally over working end 56 and over limb 115a while working end 56 preferably holds a terminal end of limb 115a. Further withdrawal of suture passer 10 may reduce tissue repair loop 120 and close the opening or tear 12. Once the tissue is in a repaired arrangement, or a target size of repair loop 120 has been reached, tension may be applied to the second limb 115b to reduce loops 130a, 130b and lock the knot 110. Flexible strand 101 may then be trimmed.

In some embodiments, at least one of the limbs 115a, 115b may include a marking, at least towards the terminal ends 110a, for easy identification. Instrument 50 may include a means to retain knot 110′ at proximal end of shaft 55. For example, a slideable O-ring 70 may be disposed around the outer surface of shaft 55, for retaining and managing the loops 130a, 130b. More specifically the second limb 115b may be operatively coupled to the O-ring 70, of pushed behind (proximal to) an O-ring 70, to manage clutter of the flexible member construct.

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

What is claimed is:

1. A loading tray for managing a flexible member construct that includes a pre-formed knot, the pre-formed knot including a loop, with a first limb and a second limb extending therefrom, the loading tray comprising:
a base; and
a knot loader hingedly coupled to the base, the knot loader defining an outer peripheral surface configured to hold the pre-formed knot loop in a dilated configuration; and wherein the knot loader also defines a channel configured to receive an instrument shaft therethrough and thereby place an instrument shaft through the pre-formed knot loop.

2. The loading tray of claim 1 wherein the knot loader is hingedly coupled at a first end of the knot loader, such that external forces from the instrument shaft while within the channel, pivots the knot loader around the first end, draws the loop over a free end of the knot loader and onto the instrument shaft.

3. The loading tray of claim 1 wherein the pre-formed knot is an incomplete sliding and locking knot.

4. The loading tray of claim 1 wherein the loading tray is entirely formed from a single unibody.

5. The loading tray of claim 1 wherein the loading tray is formed from a material selected from the group consisting of cardboard, paper, cork or a polymer.

6. The loading tray of claim 1 wherein the loading tray is formed of card and the knot loader includes angularly offset portions thereof, defining three sides of the channel.

7. The loading tray of claim 1 wherein the knot loader is hingedly coupled via a living hinge.

8. The loading tray of claim 1 further comprising a first and a second slot for holding a first and a second limb of the flexible member construct respectively.

9. A method of arthroscopically repairing a tissue using a suture passer and a loading tray with a flexible member construct assembled thereto, the flexible member construct including a pre-formed knot with at least one dilated loop and a first and second limb, the at least one dilated loop assembled around a housing of the loading tray; the method comprising:
passing a distal end of the suture passer through a channel of the housing, and thereby threading the suture passer through the at least one dilated loop;
removing the at least one dilated loop from the housing and onto a shaft of the suture passer;
operatively coupling a first limb with a working end of the suture passer;
passing the first limb through the tissue with the suture passer, while the at least one dilated loop remains on the shaft of the suture passer;
removing the at least one dilated loop from the suture passer and onto the first limb; and
reducing the at least one dilated loop to repair the tissue.

10. The method of claim 9 wherein the loading tray includes a first slot for holding the first limb and wherein operatively coupling the first limb with the working end of the suture passer comprising removing the first limb from the first slot.

11. The method of claim 10 wherein the loading tray includes a second slot for holding the second limb and therein the method further comprises removing the second limb from the second slot after passing a distal end of the suture passer through the channel and applying tension on the second limb to reduce the at least one dilated loop around the suture passer.

12. The method of claim 11 wherein removing the at least one dilated loop from the suture passer and onto the first limb changes the preformed knot configuration to a sliding-locking knot.

13. The method of claim 9 wherein passing the first limb forms a repair loop through the tissue and wherein removing the at least dilated loop changes the pre-formed knot into a locking knot configured to lock the repair loop.

14. A method of assembling a flexible member construct with an instrument shaft, the flexible member construct including a pre-formed knot with a loop and a first and second limb, the method comprising;
placing an instrument shaft through a dilated loop of the preformed knot, the dilated loop wrapped around a knot loader of a loading tray, and
draw the dilated loop over a free end of knot holder and onto the instrument shaft.

15. The method of claim 14 further comprising pulling on at least one of the first and second limb to draw the dilated loop over the free end of the knot holder.

16. The method of claim 14 further comprising lifting the suture passer placed through the dilated loop away from the knot loader to pivot a hinge of the knot loader and draw the dilated loop over the free end of the knot holder.

17. The method of claim 14 further comprising tensioning one of the first or second limb to tighten the dilated loop around the instrument shaft.

18. The method of claim 14 further comprising operatively coupling the first limb to a working end of the suture passer.

19. The method of claim 14 wherein the first and second limbs are both operatively coupled to the loading tray and wherein the method further comprises removing the first limb from the loading tray and operatively coupling the first limb to a working end of the suture passer, and also comprises removing the second limb from the loading tray and pulling on the second limb to reduce the dilated loop.

20. The method of claim 14 wherein placing the instrument shaft through a dilated loop comprises inserting the instrument shaft through a channel of the knot loader.
